(19) United States
(12) Patent Application Publication
(10) Pub. No.: US 2007/0219542 A1
(43) Pub. Date: Sep. 20, 2007

(54) Surgical procedure and instrumentation for intrastromal implants of lens or strengthening materials

(76) Inventor: Toru Yahagi, Tokyo (JP)

Correspondence Address: RONALD CRAIG FISH, P.O. BOX 820, LOS GATOS, CA, 95031, US

(21) Appl. No.: 11/377,087

(22) Filed: Mar. 15, 2006

(51) Int. Cl. A61B 18/18

(52) U.S. Cl. 606/5

(57) ABSTRACT

A surgical procedure for implanting a lens or strengthening material in a cornea without making a flap as is done in Lasik procedures. A femtosecond laser or some future developed laser which has the same ability is modified to cut a pocket in the stroma layer of a cornea with no side cuts and only an opening at the surface of a cornea. Alternatively, a modified microkeratome shaped and sized to form the desired shape for a passageway bed cut which is cut to join a central bed cut. Alternatively, the passageway bed cut can be done using a femtosecond laser or some future developed laser which has the same ability. The passageway bed cut is smaller in dimension than a central bed cut of a pocket in which the lens will be implanted in procedures where an expandable lens or strengthening material is used. A modified microkeratome having a web, a central blade shaped to form the passageway bed cut and sled runner on either side of the blade and separated therefrom is also disclosed. A patient interface with a mask is also disclosed to allow a femtosecond laser or some future developed laser which has the same ability may also be used to form both the central bed cut and a passageway bed cut or only a single bed cut with an opening at the surface of the cornea to form the pocket is also disclosed.

### BACKGROUND OF THE INVENTION

The popularity of refractive surgery skyrocketed when surgical laser was introduced in 1980s. However, even the latest laser technology has its shortcomings. While laser is very effective in reducing the thickness of cornea with extremely high precisions in correcting refractive error, it has been useless against diseases caused by poor the structural integrity of cornea. Such diseases include Keratoconus and Corneal Ectasia. Keratoconus is insufficient corneal strength to withstand the intraocular pressure thereby causing bulging and thinning of the cornea. Corneal Ectasia is insufficient corneal strength to withstand the intraocular pressure causing bulging of the cornea and results from over thinning of the cornea during refractive surgery. This is a common complication of popular refractive surgeries. A surgical laser can never strengthen the cornea. It is therefore desirable that in any new surgical procedure to treat either Keratoconus or Corneal Ectasia, it is desirable to not further weaken the structural integrity of cornea.

Keratoconus is a progressive condition which is characterized by thinning and bulging of the cornea. When the abnormally shaped cone-like cornea resulting from Keratoconus becomes pronounced, it impairs one's eyesight because of refractive error exceeding the power of the crystalline lens to focus. Typically, the disease starts between the late teens and the early twenties. In earlier stages of Keratoconus, rigid gas permeable contact lens can be used to correct refractive error. In more advanced stages of Keratoconus, patients usually become heavily myopic and have a high degree of astigmatism that is not correctable with glasses or contact lens. Currently, corneal transplant is the only treatment option in the more advanced stage of Keratoconus. Corneal Ectasia is a condition very similar to Keratoconus. It is one of the complications caused by refractive surgeries in which the cornea becomes too thin to withstand the intraocular pressure. To maintain the structural integrity of cornea integrity and avoid Corneal Ectasia, the surgeons are to adhere to certain rules for the minimum thickness of cornea as a result of refractive surgery procedures. This condition may also call for corneal transplants. This surgery is expensive and, like all surgery, has risks. Further, donor corneas are hard to come by, especially in some countries. Therefore, a need has arisen for a surgical procedure and apparatus to carry out such a procedure to eliminate the need for corneal transplants.

One disadvantage of refractive surgery is the fact that it is irreversible because corneal tissue of the stroma layer is removed in the ablation process and the stroma layer does not regenerate. One problem arising from this irreversibility is that the focal power of the eye declines with age. Aging results in stiffening of the crystalline lens and weakening of the ciliary muscles that change the thickness of the crystalline lens to focus. Stiffened crystalline lens and weakened ciliary muscles become ineffective in focusing and cause presbyopia. Thus, a person who has refractive surgery early in life develops presbyopia later without the ability to have it reversed. Another problem with prior art refractive surgeries is that physicians are left with few options once the patient's refractive error is overcorrected. It is physically impossible to undo what has been done to the stroma layer of cornea once laser ablation removes corneal tissue.

Another disadvantage of refractive surgery is that it has a limited ability to correct refractive error. In modern refractive surgeries where excimer laser devices are used, laser ablates the cornea to make it thinner. In extreme cases, it requires more tissue to be removed than possible to achieve the desired refractive correction without weakening the structural integrity of cornea. The cornea is about 500 microns thick with 95% of that thickness being the stroma layer (which does not regenerate) and the rest being epithelium layer, Bowman's membrane, Descemet's membrane and the endothelium. The rule of thumb is that when performing ablation in refractive surgeries, at least 250 microns of stroma tissue called stroma “bed” must be left after the procedure to ensure adequate structural integrity. Thus, there are limits on how large of refractive correction can be done.

The Stanford University Corneal Product Development Team of the Stanford Department of Ophthalmology and Department of Chemical Engineering has developed a corneal inlay procedure for refractive correction. In this procedure, a prior art LASIK flap is created. A flap can be created with a microkeratome or a laser device, but in both cases, the flap weakens the structural integrity of cornea.

The applicants believe this flap in the Stanford procedure is created in the same way as such LASIK flaps are created in prior art LASIK procedures. Specifically, the applicants believe a microkeratome is used in the Stanford procedure to create the flap. The flap may be lifted up to expose the underlying the stroma layer much like the lid of a copy machine is lifted up and tilted back. A polymeric lenticule is then placed on the stroma layer. It is held in place laterally on the cornea (orthogonal to the focal length axis of the eye) not by any physical restraint provided by stroma tissue but by surface tension between the surface of stroma under the flap and the lenticule. The flap is then laid back down over the lenticule, and the epithelium layer regenerates quickly to seal the flap and hold the lenticule in place.

Stanford has one issued U.S. Pat. No. 6,976,997 covering the structure of artificial cornea implants, and methods for making and using such implants.

Therefore, a need has arisen for surgical procedure and instrumentation to practice it in order to correct refractive error using an intrastromal implant in a procedure which is reversible and which does not reduce the structural integrity of cornea by creating a flap. In addition, implanting extra material inside of the stroma enhances the structural integrity of cornea, preventing further distortion of the cornea in Keratoconus or Corneal Ectasia.

### SUMMARY OF THE INVENTION

According to the teachings of the invention, a “pocket” is formed in the cornea, and a lens or strengthening material is inserted into the pocket, the choice of implant depends upon the condition being treated. A “pocket” is a location inside the stroma layer formed by one or more cuts made in any way (by blade or laser) which has an opening at the surface of the cornea into which the lens or strengthening material will be inserted. The pocket also has as a part thereof a “central bed cut” which is the portion of the pocket where the lens or strengthening material rests in the final configuration after the surgery is completed. A “central bed cut” is a cut made inside the stroma layer by a femtosecond laser or some future developed laser which has the same ability. The “central bed cut” does not intersect the surface of the cornea at any point along its perimeter as it has no “side cuts”. A “side cut” is a cut made by a laser which intersects both the surface of the cornea and the central bed cut and is generally parallel to the Y axis in an X-Y Cartesian coordinate space where the Y axis is orthogonal to the plane a microkeratome would lie in when cutting a flap for traditional refractive correction surgery (hereafter the blade plane), and the X axis lies in the blade plane.

The pocket is formed by making cuts which weaken the structural integrity of cornea less than the cuts to make a flap. Specifically, in one example, the pocket is made by first making a “central bed cut” in the stroma layer of a cornea and then cutting a “passageway bed cut” to join the central bed cut. The passageway bed cut has an opening at one end which intersects the surface of the cornea. The opening is the location where the lens or strengthening material is inserted and must be suitably sized to fit the original size of the lens or strengthening material. In some examples within the teachings of the invention, the lens or strengthening material can be expandable. The lens or strengthening material is slipped into the passageway and slid into the portion of the pocket formed by the “central bed cut”.

LASIK flaps, which are not necessary in the invention, are used in LASIK procedures and the prior art Stanford intrastromal inlay procedure. LASIK flaps are formed in either of two ways: 1) with a femtosecond laser which makes a bed cut in the X axis and then makes side cuts which intersect the bed cut and also intersect the surface of the cornea; 2) with a microkeratome. All LASIK flaps have a prior art LASIK hinge portion and cuts to the surface of the cornea on all portions of the perimeter of the flap other than the LASIK hinge portion. The presence of these cuts to the surface of the cornea along the non-LASIK hinge portions of the flap weakens the structural integrity of cornea.

In examples which use two or more bed cuts to form the pocket, the passageway bed cut is at the same level in the stroma layer as the central bed cut and parallel to the plane thereof. However, in some examples, the passageway bed cut can angle down from the surface of the cornea to terminate at the level of the central bed cut somewhere in or at the edge thereof so as to form a passageway for the lens or strengthening material into the central bed cut.

If a procedure according to the invention is being used to treat, Keratoconus and Ectasia, the material inserted into the pocket need not provide refractive correction and only needs to strengthen the cornea. If the condition being treated is both Keratoconus or Corneal Ectasia and some refractive error, the implant can be a lens or strengthening material which both provides refractive correction and additional structural integrity to prevent bulging by the forces causes by the intraocular pressure.

In some examples, the central bed cut is big enough to hold a non-expandable lens or strengthening material. In other examples, a central bed cut is cut which is big enough to hold the lens or strengthening material after expansion by absorption of water, and a passageway bed cut which is only big enough to fit the non-expanded lens or strengthening material is cut so as to join the central bed cut.

The function of the central bed cut is to provide a mechanical mounting for the lens or strengthening material to hold it in place. In the preferred example, the lens or strengthening material is a double network hyrdrogel with high permeability to oxygen and glucose and with high strength and elasticity.

If the patient is being treated for refractive error, the lens will have a shape which corrects the refractive error. If the procedure is used to treat only Keratoconus or Corneal Ectasia, the implant need not have any refractive error correction properties but, it must have sufficient structural strength to reinforce the cornea and add to its structural integrity to combat bulging out of the cornea due to the intraocular pressure. If the procedure is used to treat either Keratoconus or Corneal Ectasia as well as visual acuity properties, the implant needs to have refractive error correction properties as well as sufficient structural strength to reinforce the cornea enough to add to its structural integrity to combat bulging out of the cornea under the intraocular pressure.

One advantage of the procedure according to the teachings of the invention, is that as the patient ages and the crystalline lens stiffens and the ciliary muscles that change the shape of the crystalline lens weaken, the lens or strengthening material previously implanted can be removed or a new lens or strengthening material substituted. This means the patient will still be able to focus over a broad range of distances to the subject.

In the Detailed Description of the Invention given below, there are several examples of different shapes for the central bed cut and passageway bed cut. These examples are: Example 1 comprising the three cut embodiment of FIG. 10 (central bed cut, passageway bed cut and side cut for opening) where the passageway bed cut is wider than the diameter of the central bed cut; Sample 2 comprising the three cut embodiment of FIG. 11 where the passageway bed cut is the same width as the central bed cut; Sample 3 comprising the three cut embodiment of FIG. 12 where the passageway bed cut is a truncated triangle shape; Sample 4 comprising the three cut embodiment of FIG. 13 where the passageway bed cut is a narrow rectangular bed cut which is much narrower than the diameter of the central bed cut; and Sample 5 comprising the one or two cut embodiment of FIGS. 14A and 14B (one or two cuts depends upon whether the cut is made with a laser device or microkeratome). Sample 1 through 4 can all be made using a prior art Intralase femtosecond laser which has been modified to use a mask to control cutting of the passageway bed cut to get the desired shape. Sample 1 through 4 can all also be made using a laser device which does not exist yet but which will have at least the following characteristics: 1) the laser must generate a beam of energy which can pass through the cornea tissues without harming them if the beam is not focused; 2) the operator must be able to focus the beam at a desired spot and at a desired depth within said stroma; 3) the energy level at said focal point for said beam must rise to the level of ablation; 4) the laser must be able to generate a very short burst of energy at the focal point which is of sufficient duration to create the desired ablation, but not so long as to do excessive damage; and 5) the shape and position of the lines of pixels of ablation created by the laser must be controllable in any way. Sample 5 of FIG. 14A can only be created by this future developed femtosecond laser (because it is a two cut embodiment—combined central bed cut and passageway bed cut as one cut plus a side cut for the opening into the passageway). Sample 5 of FIG. 14B can also be created using a modified microkeratome as a one cut embodiment where the single cut forms both the combined central bed cut and passageway bed cut as well as the opening. Passageway bed cut in sample 2 and 4 may also be formed by specially shaped microkeratome.

In the Detailed Description given below, the term “Intralase FS laser” is used liberally. The term should be interpreted to mean not only prior art femtosecond lasers manufactured by Intralase, but also femtosecond lasers having the above given five characteristics including the ability to control the pattern and position of ablation pixels created by the laser. Further, it should be understood that the prior art Intralase FS laser can be modified to make the central bed cut and passageway bed cuts of the various examples either by using a mask as described for Sample 1 and 2 but also, if access to the laser's source code is available, by modifying that source code to enable control over the shape and position of the pixe patterns created.

### BRIEF DESCRIPTION OF THE DRAWINGS

FIG. 1 is a diagram of the prior art human eye.

FIG. 2 is a diagram of the prior art natural cornea.

FIG. 3 is a diagram showing how the eyelid is clamped open as a first step in the procedure.

FIG. 4 is a diagram showing how an Intralase patient interface suction is attached to the eye to stabilize it.

FIG. 5 is a top view of the shape of the central bed cut which houses the lens or strengthening material.

FIG. 6 is a cross-sectional view of the cornea showing a typical position of the central bed cut in one example.

FIG. 7 is a top view of the prior art LASIK flap showing the typical side cut configuration.

FIG. 8 shows a top view of the laser mask which is used in one example of a surgical procedure according to teachings of the invention to make the necessary bed cut shape and side cut to form a passageway through which the lens or strengthening material can be inserted into the bed cut 34.

FIG. 9 shows the outline of the passageway bed cut and a side cut which serves to form an opening into which the lens or strengthening material is inserted.

FIG. 10 shows the final configuration of the passageway bed cut and its overlap with the central bed cut and the opening at the surface of the cornea into the passageway bed cut.

FIG. 11 shows the most preferred configuration for the central bed cut and passageway bed cut and opening of the passageway bed cut.

FIG. 12 is a top diagram looking down into the cornea of the outlines of another example having a central bed cut like the examples previously described, and a narrower passageway bed cut for use with an expandable lens or strengthening material for implant 62.

FIG. 13 is a top diagram looking down into the cornea of the outlines of another example having a central bed cut like the examples previously described, and a narrower passageway bed cut for use with an expandable lens or strengthening material for implant 62.

FIGS. 14A and 14B are diagrams of a specially shaped bed cut made using an example of the invention where the bed cut combines the shapes of the central bed cut and the passageway bed cut. The bed cut of FIG. 14A is made with a femtosecond laser or future laser with the same capability as a femtosecond laser and which is programmed to make the specially shaped bed cut of FIG. 14A and the side cut 88A. The bed cut of FIG. 14B is a bed cut made by the specially shaped microkeratome of FIG. 16. If a laser is used, side cut 88A is needed because the specially shaped bed cut does not intersect the surface. If a microkeratome is used, only one cut is needed since the single cut does intersect the surface of the cornea and forms the opening of the pocket at 88B.

FIG. 15 is a diagram of a microkeratome suitable to make the passageway bed cut 80 and the opening 78 in FIG. 13.

FIG. 16 is a top view of a microkeratome suitable to make the specially shaped bed cut in the example of FIG. 14B and opening 88B.

FIG. 17 is a diagram of the prior art Intralase patient interface used in one example of the invention and which has been modified by the inclusion of a mask for purposes of making passageway bed cuts using a femtosecond laser or some future developed laser which has the same capability as a femtosecond laser.

FIG. 18 is a diagram of the chemical structure of the first network of the preferred prior art hydrogel developed by Stanford, and FIG. 19 is a diagram of the chemical structure of the second network of the preferred prior art hydrogel developed by Stanford.

FIG. 20 is a diagram of the chemical structure of the bioadhesive material that coats the hydrogel of the preferred prior art material for the lens or strengthening material developed by Stanford.

### DETAILED DESCRIPTION OF THE PREFERRED AND ALTERNATIVE EXAMPLES

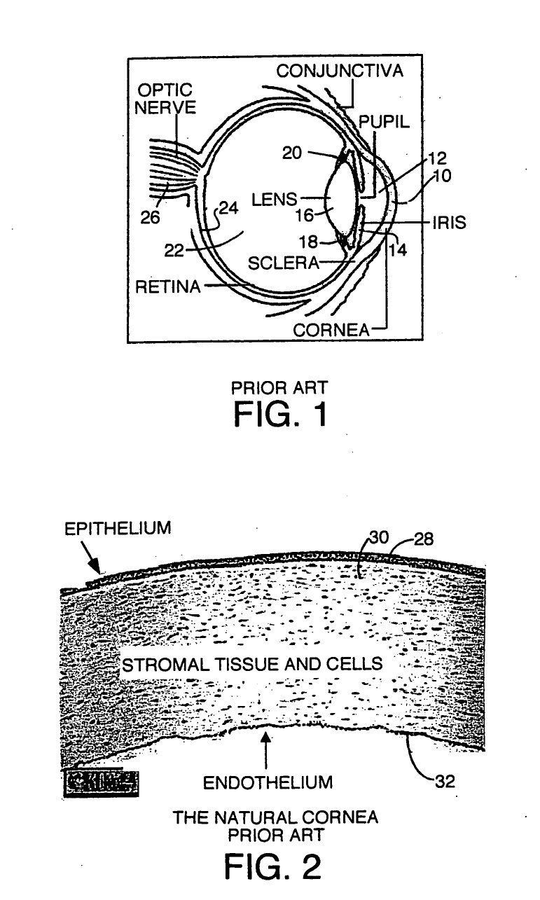

A schematic diagram of the human eye is shown in FIG. 1. The cornea 10 is the site where the surgical procedure will be performed. The cornea lies in front of the aqueous humor 12, the iris 14 which regulates the amount of light then enters the eye and the crystalline lens of the eye 16. The ciliary muscles that stretch the crystalline lens to change its shape are shown at 18 and 20. The vitreous humor 22 fills the eyeball and is a jelly-like substance which gives the eyeball its firmness. The retina is shown at 24 and lines the back of the eyeball. The retina is where the focused image is supposed to be if everything is perfect. The retina's rods and cones (not shown), sense the light pattern and send nervous signals to the brain on the optic nerve 26. The intraocular pressure exists in the area of the vitreous humor 12.

FIG. 2 is a diagram of the prior art natural cornea. The cornea is covered with a thin layer 28 called the epithelium. This layer is 4-5 cell layers thick and regenerates very fast when damaged. This layer must be supplied with glucose from the blood stream. This glucose is transported through the stroma layer 30 from blood vessels elsewhere in the eye so any device implanted in the stroma layer should be permeable to glucose traveling toward the epithelium. Just beneath the epithelium 28 and above the stroma layer 30 is Bowman's membrane (not shown).

Under the epithelium is the stroma layer 30. This layer is about 500 microns thick, and any implant or other procedure should leave at least 250 microns of the stroma tissue left to provide adequate structural support to withstand the intraocular pressure. The stroma is transparent, does not regenerate, has no blood vessels in it and is permeable to oxygen and glucose. The stroma is comprised of collagen fibrils that run parallel to each other.

Under the stroma layer is Descemet's membrane (not shown) followed by the endothelium 32. The endothelium layer is only one cell layer thick. The endothelium 32 pumps water from the cornea thereby keeping it clear. If damaged or diseased, endothelium 32 will not regenerate.

### The Surgical Procedure

The first two steps in surgical procedures according to the invention are the same as in conventional LASIK procedures. First, the eyelid is clamped open with a speculum as shown in FIG. 3. Second, the eye is stabilized to prevent movement. That can be done in any way. For example, an Intralase patient interface suction (or any device which can serve the same purpose) is attached to the eye to stabilize it and prevent movement, as shown in FIG. 4. In the preferred example, the “patient interface” stabilizes the eyeball to prevent movement and is also used to flatten the eye if the Intralase laser is used. The conventional step of flattening the cornea can be performed in any way.

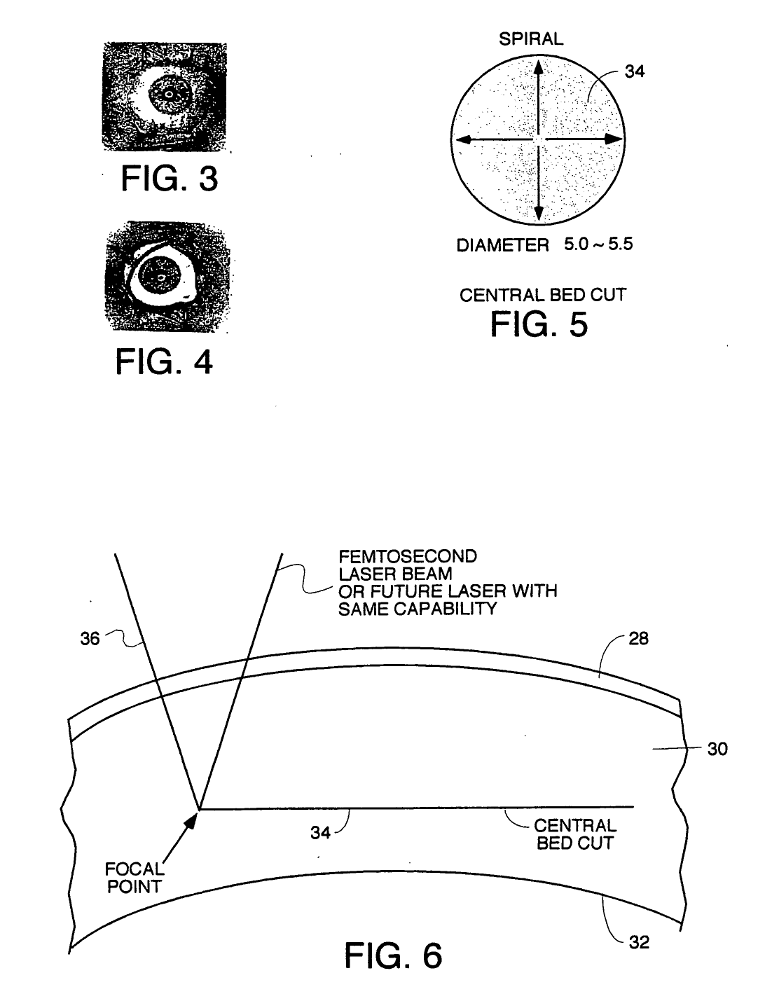

FIG. 5 is a top view showing the shape of the “central bed cut” which is cut into the stroma layer using a femtosecond laser or some future developed laser which has the same ability. A “central bed cut” is the portion of the pocket where the lens or strengthening material will rest in the final configuration after the surgery is completed. A “central bed cut” is a cut made inside the stroma layer by a femtosecond laser or some future developed laser which has the same capabilities. The “central bed cut” does not intersect the surface of the cornea at any point along its perimeter as it has no “side cuts”. The purpose of the central bed cut is to form the portion of the pocket in the stroma layer into which the lens or strengthening material is held. The central bed cut is a circular cut having a diameter of approximately 5.0 to 5.5 millimeters in the preferred example. It is made using a femtosecond laser or some future developed laser which has the same ability and which is programmed to cut in spiral mode and results in ablation of a slit or pocket inside the stroma layer with no opening to the surface of the cornea. A passageway bed cut that intersects the central bed cut will need to be formed which will have an opening at the surface of the cornea into which the lens or strengthening material will be inserted. The lens or strengthening material will then be slid through the passageway into the central bed cut.

FIG. 6 shows a typical position of a typical central bed cut 34 in the stroma layer 30. A minimum of about 250 microns of the stroma tissue beneath the bed cut is preferred to maintain structural strength. The central bed cut is formed by focusing the laser beam of a femtosecond or similar capability laser at the desired level of the bed cut. It is focused at the focal point 37 which is the level in the stroma layer where the bed cut is to be made. The focusing of the beam at the focal point causes the stroma tissue at the focal point to be ablated. The central bed cut is made by shooting computer controlled microbursts of laser energy from a femtosecond laser or some future developed laser which has the same ability which is programmed to shoot “pixels” or bursts of focused energy in a spiral pattern having a circular outer diameter that is about 5.0 to 5.5 millimeters. The depth of the bed cut in the stroma layer from the epithelium depends upon such variances as the thickness of the cornea, the arc of the cornea and type of material to be inserted.

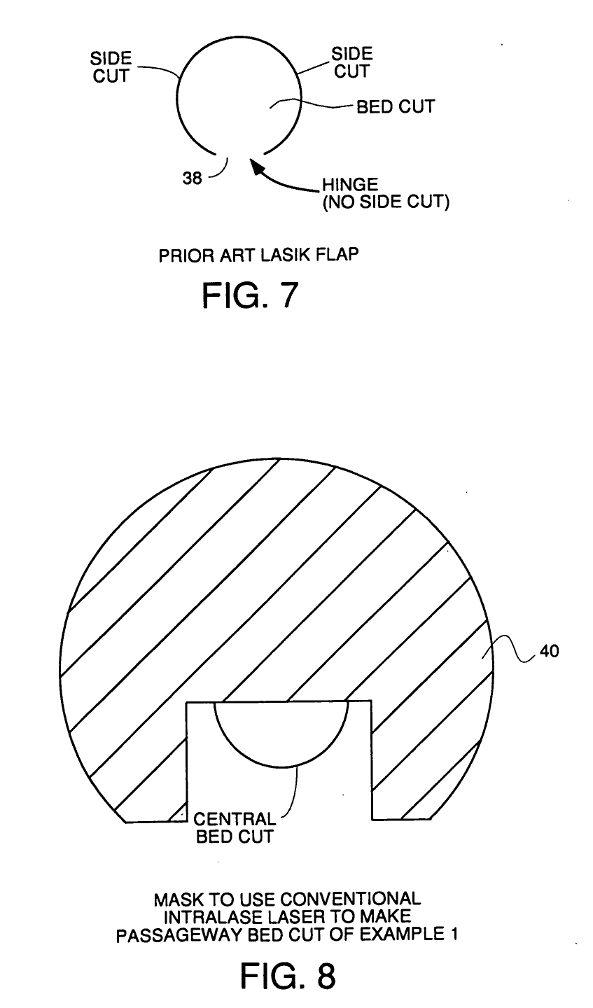

At this point, the bed cut 34 shown in FIG. 5 has no side cuts. As such, it is different from the flap in a LASIK procedure. In a LASIK flap, it is the circular side cuts that intersect with the surface of the cornea and the bed cut which leaves the LASIK hinge portion 38 of the flap intact. It is the “LASIK hinge” (the portion of the perimeter of the flap with no side cuts) and the side cuts in the LASIK flap which allows the flap to be lifted up so that laser ablation of the underlying stroma tissue can occur in the prior art LASIK procedure. FIG. 7 shows a top view of a prior art LASIK flap. A bed cut in the prior art LASIK procedure is a cut made inside the stroma layer by a femtosecond laser or some future developed laser which has the same ability. The bed cut of the prior art LASIK flap does not intersect the surface of the cornea at any point. The side cut needed to form the prior art LASIK flap goes from the surface of the cornea down to the “bed cut” and extends around the entire perimeter of the bed cut except for the “LASIK hinge” area 38.

The Stanford Corneal Inlay prior art procedure also creates a corneal flap with side cut. This flap is lifted up to allow the hydrogel corneal inlay to be placed under the flap.

FIG. 8 shows a top view of the laser mask which is used to make the necessary passageway bed cut and side cut to form the opening through which the lens or strengthening material can be inserted into the central bed cut 34. The mask 40 is made of paper, plastic or some other material which is opaque to the femtosecond laser or some future developed laser which has the same ability. The purpose of the mask 40 is to prevent laser beam from reaching any tissue below the masked area. This mask would not be necessary if the femtosecond laser or some future developed laser is modified to make the desired pattern for the central bed cut and passageway bed cut and the opening. Since existing femtosecond laser devices are not yet designed to create such desired cuts, this mask is necessary when using the unmodified prior art Intralase laser.

The manner in which the unmodified Intralase FS laser is used to do surgical procedures according to the teachings of the invention is as follows. In the prior art Intralase FS laser, there is control by the operator over only four parameters: 1) diameter of the flap; 2) location of the LASIK hinge; 3) spiral or raster cutting mode; 4) depth of the bed cut. The central bed cut is made first by setting the diameter at about 5.0 to 5.5 millimeters or whatever the desired diameter for the lens or strengthening material to be inserted is. The depth parameter is set at a depth suitable for the condition being treated. The cutting mode parameter is typically set to spiral mode and the location of the LASIK hinge can be anywhere because the operation is aborted before cutting of the side cuts occurs. In the prior art Intralase FS lasers, the operator has a footpedal which he or she can push to start and stop the automatic cutting process at any point. The Intralase FS laser first cuts the bed cut, and then starts making the side cut around the perimeter of the bed cut to form the prior art flap. The operator using the Intralase FS laser to do the procedure of the invention stops the Intralase FS laser after it has made the central bed cut and before it starts making the side cut. That completes the making of the central bed cut.

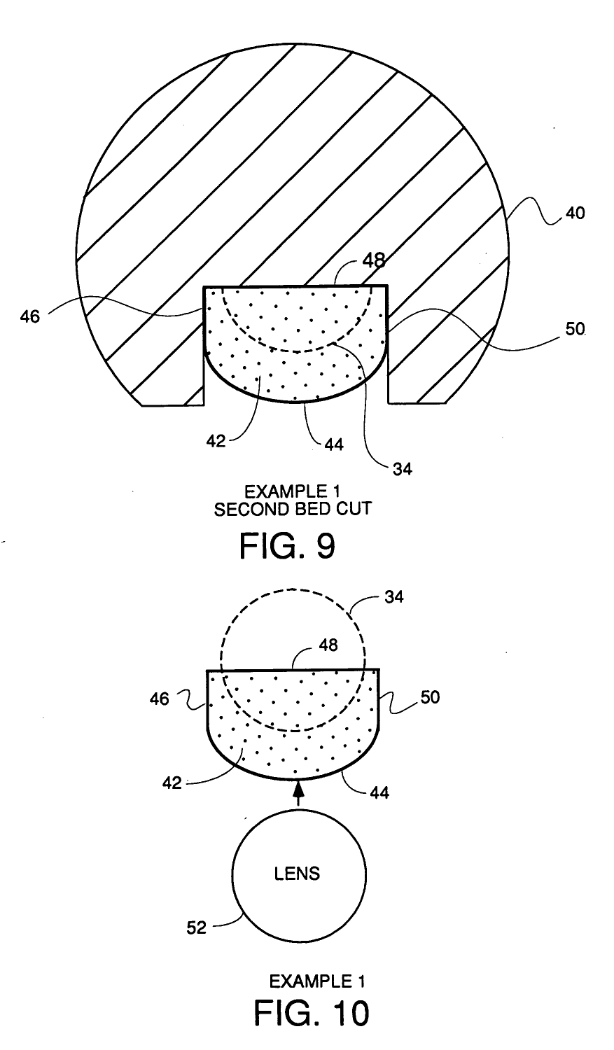

Next, to make the passageway bed cut, a mask shape shown at 40 in FIG. 8 is inserted on the lower glass portion 104 in FIG. 17 of the Intralase patient interface already engaged with the eye. An Intralase patient interface with such a mask in place is shown in FIG. 17. Then, the diameter parameter of the bed cut is changed to the diameter of a circle having the side cut 44 in FIG. 9 (which will ultimately serve as the opening to the passageway bed cut) as a portion of its perimeter. The depth parameter of the bed cut for the passageway bed cut is set to be the same as the depth of the previously formed central bed cut in this example. In other examples using a modified Intralase laser which can cut sloping bed cuts, the passageway bed cut can be cut at a slope from the surface of the cornea down to intersect the central bed cut so as to eliminate a side cut orthogonal to the surface of the cornea. The position of the LASIK hinge is then specified to be anywhere on the perimeter of the circle of the passageway bed cut which is under the mask 40. The cutting mode is set to raster. The cutting process is then started. The Intralase laser then tries to cut a flap having the diameter of the circle which includes opening 44 in its perimeter. However, because of the presence of the mask 40, it only actually cuts a passageway bed cut 42 having the shape the stippled portion of FIG. 9 and an opening 44 which is arcuate. The laser will trace a raster pattern, but the mask 40 will block the beam from reaching any tissue under the mask 40 so only the stippled portion in FIG. 9 will actually be cut to form the passageway bed cut 42. The mask 40 prevents any cutting of any tissue outside the stippled portion 42 which is under the mask 40. The operator then allows the Intralase laser to start making the side cut. The laser will attempt to make a side cut, but only the opening 44 will be cut. The side cut portions which would have occurred but for the presence of mask 40 will be attempted by the laser but not actually cut since the laser beam never reaches any tissue under mask 40. This leaves the passageway bed cut 42 and opening 44 to complete formation of the central bed cut 34, passageway bed cut 42 and opening 44 shown in FIG. 9.

Use of the physical mask 40 is only one example. In other examples, a femtosecond laser or some future developed laser which has the same ability will be modified to cut the shape defined by the heavy lines 46, 48, 50 and 44 in FIG. 9 to form a passageway bed cut 42. The same is true for all examples disclosed herein. The portion of the software that defines the outline of the bed cut shape can be modified to cut any of the shapes described herein for the central and/or passageway bed cut.

The passageway bed cut formed using mask 40 has a shape defined by the heavy lines 46, 48, 50 and 44 and the stippled area 42. The passageway bed cut has a diameter of approximately 9.0 to 9.5 millimeters preferably for the circle whose perimeter includes line 44.

FIG. 10 shows the final configuration of the passageway bed cut and its overlap with the central bed cut. A side cut is made along line 44 so that lens or strengthening material 52 may be inserted into the central bed cut 34 through the opening 44 and the passageway bed cut 42. Suitable materials for the lens or strengthening material will be discussed below. After the lens or strengthening material is inserted, the procedure is finished and the epithelium will regenerate over the opening 44 and seal the lens or strengthening material in the central bed cut 34.

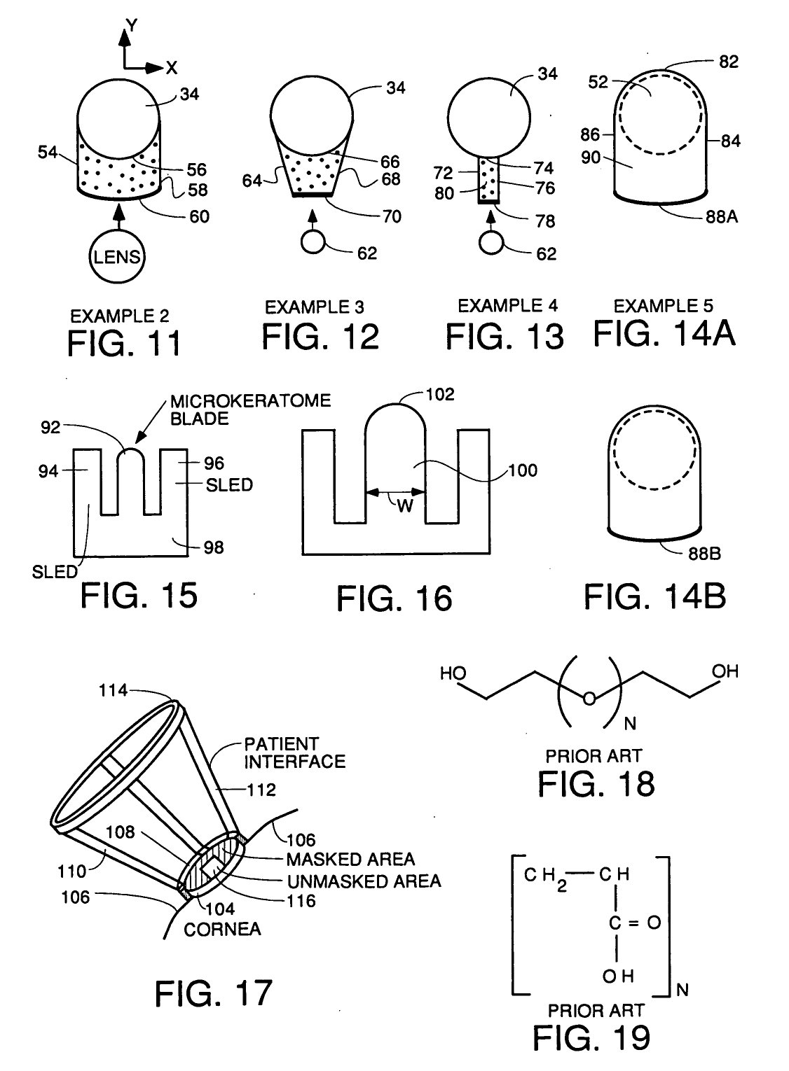

FIG. 11 shows the most preferred example for the central bed cut and passageway bed cut and the opening of the surgical procedure. In this example, the central bed cut 34 is made in the same manner and using the same equipment as described above in discussing FIGS. 5 and 6. The passageway bed cut is preferably made at the same level in the stroma layer as the central bed cut and has the shape defined by lines 54, 56, 58 and 60. Line 54 and 58 do not represent side cuts. Only line 60 represents a side cut to the surface of the cornea so as to form the opening into the pocket. This shape will be referred to in the claims as a “passageway”. Line 56 defines the boundary where the central bet cut joins the passageway bed cut. In some examples, only the joinder boundary 56 where the passageway bed cut joins the central bed cut needs to be at the same level in the stroma layer as the central bed cut. The rest of the passageway bed cut can angle down into the stroma layer from the surface of the cornea so as to terminate at the level of the central bed cut and intersect the central bed cut within or at its perimeter so as to allow a lens or strengthening material to be inserted through the opening and the passageway bed cut into said central bed cut.

FIG. 17 is a diagram of the Intralase patient interface with a mask for purposes of making the passageway bed cut and the opening using a femtosecond laser or some future developed laser which has the same ability. An Intralase patient interface has a transparent eye contact plate portion 104 (typically made of glass or some other rigid transparent material). The eye contact plate is pressed against the cornea 106 and flattens the cornea slightly at the location of contact. The eye contact plate is surrounded by an frame 108 made of aluminum or some other sufficiently strong metal or plastic material which can be attached to the eye contact plate to support it and to act as a means to attach the eye contact plate to multiple legs which extend away from the plane of the eye contact plate. To this frame there are attached multiple legs of which legs 110 and 112 are typical. The legs couple the eye contact plate frame 108 to an upper ring frame 114 which can be attached to the A femtosecond laser or some future developed laser which has the same ability. The output beam of the laser is directed through the middle of the upper ring 114 through the transparent eye contact plate 104.

The teachings of one aspect of the invention contemplate placing a mask which is opaque to the laser energy on the top surface of the eye contact plate 104 (the surface not touching the cornea) and which is patterned to have an opaque portion and a transparent portion. The mask could also be placed between the eye contact plate and the upper ring 114. The important thing is that the mask be removable. This is because the Intralase patient interface is placed in contact with the cornea only once to make both the central bed cut and the passageway bed cut. If the Intralase patient interface were to be removed after making the central bed cut and a new Intralase patient interface with a mask were to then be substituted and re-engaged with the eye, the compression caused by the first patient interface-cornea contact could not be precisely duplicated in the second patient interface-cornea contact. This would be undesirable because the compression is important to the depth of the cut and the depth of the central bed cut and the passageway bed cut must be precisely controlled to be the same. Thus, to use the patient interface, a Intralase patient interface with no mask is brought into contact with the cornea and compresses it and the central bed cut is made. Then, the mask is placed somewhere in the Intralase patient interface so as to block the laser beam appropriately to make the desired shape for the passageway bed cut, and the parameters of the Intralase FS laser or other suitable laser are adjusted as previously described and the passageway bed cut is formed.

The transparent portion 116 of the mask has a shape which is the desired shape of the passageway bed cut to be made with the laser. The laser beam is focused so that its focal point is down in the stroma layer at the level of the desired cut. The energy level rises at the focal point enough to vaporize the stroma tissue during every microburst. By making thousands of tiny pixel cuts like this, a bed cut may be made without ever cutting through the surface of the cornea because the unfocussed laser energy passing through the surface of the cornea does not have sufficient energy to vaporize epithelium or stroma tissue. An unmodified femtosecond laser or some future developed laser which has the same ability and which is set up to do a spiral or raster cut mode through an Intralase patient interface modified in the manner shown in FIG. 17 may be used to perform the passageway bed cutting steps of the various sample of the surgical procedure. The transparent portion of the mask is altered according to the desired bed cut shape of the sample being practiced to form the desired passageway bed cut.

The example of FIG. 11 is preferred for non-expandable lens or strengthening material 52. This preference over the example of FIG. 10 is because the passageway bed cut is the same width as the central bed cut thereby reducing the possibility that the lens or strengthening material can move along the X axis and get misaligned with the intersection 56 with the central bed cut.

A preferred example of a surgical procedure according to the teachings of the invention for use with non-expandable lens or strengthening material is shown in FIG. 14A. In this example, only a single bed cut is performed using a microkeratome. The bed cut has the shape defined by lines 82, 84, 86 and 88A. This shape will be referred to in the claims as the “rounded shirt pocket shape”. Lines 86 and 84 do not represent side cuts—they are the boundaries of the bed cut and lie down inside the stroma layer and do not intersect the surface of the cornea. None of the boundaries 82, 84 or 86 of the bed cut intersect the surface of the cornea. There is a boundary of the bed cut under side cut 88A but it lies beneath the opening formed by making side cut 88A and is not separately shown. A femtosecond laser is programmed to make a side cut 88A which intersects the bed cut and forms an opening at the surface of the cornea so that the implant may be slid through the opening 88A into the shirt pocket shaped bed cut.

FIG. 14B represents the single cut bed cut example of the teachings of the invention where the central bed cut and passageway bed cut and opening 88B are all formed in a single cut performed by a microkeratome shaped as shown in FIG. 16.

In single bed cut examples, instead of making a separate central bed cut and passageway bed cut, only a single bed cut having the shape defined by lines 82, 84, 86 and 88B is formed. Line 88A represents the side cut pocket opening in the laser example where the side cut opening 88A is cut by the laser down from the surface of the cornea to join the bed cut. This side cut forms an opening into a passageway for a non-expandable lens material to be inserted 52. The lens or strengthening material 52 occupies the position indicated by the dashed lines after insertion. Because the passageway in the examples of FIGS. 11 and 14A and 14B is as wide as the lens or strengthening material, there is the possibility that the lens or strengthening material can shift along the Y axis, and this is undesirable.

The examples represented by FIGS. 12 and 13, are preferred examples that reduce the possibility of shifting of the lens or strengthening material after insertion by using a specially shaped cut and an expandable lens or strengthening material such as the Stanford University double network hydrogel lenticular. FIG. 12 is a top diagram looking down into the cornea of the outlines of a preferred sample for use with a flexible, expandable lens or strengthening material 62. This sample has a central bed cut like the examples previously described in FIGS. 11 and 10, but has a much narrower passageway bed cut for use as a passageway through which the expandable lens or strengthening material 62 is inserted into the pocket formed by the central bed cut 34.

FIG. 13 is a top diagram looking down into the cornea of the outlines of another example having a central bed cut like the examples previously described in FIGS. 10 and 11, and a narrower passageway bed cut for use with an expandable lens or strengthening material for implant 62.

The idea of the examples of FIGS. 12 and 13 is to insert the expandable lens or strengthening material 62 into the central bed cut through a narrower passageway bed cut. In FIG. 12, the passageway bed cut is represented by lines 64, 66, 68 and 70. In FIG. 13, the passageway bed cut is defined by lines 72, 74, 76 and 78. After insertion, the lens or strengthening material starts to absorb water from the stroma layer and expands to the approximate size of the central bed cut 34.

The advantage of the procedure represented by FIGS. 12 and 13 is that a narrower passageway bed cut is made to join with the central bed cut, and, after the lens or strengthening material is inserted into the central bed cut, through the passageway bed cut, it expands to the size of the central bed cut. The implant is therefore constricted from further movement by the walls of the central bed cut and the narrower size of the passageway bed cut.

Referring specifically to FIG. 12, the central bed cut 34 is formed using an A femtosecond laser or some future developed laser which has the same ability in the same manner as previously described in connection with FIGS. 5 and 6, preferably operating in spiral cut mode. The passageway bed cut has a shape defined by lines 64, 66, 68 and 70 and has what will be referred to in the claims as a truncated triangle shape with one end of the truncated triangle having the same width in the X axis as the diameter of the central bed cut 34. Lines 64 and 68 do not represent side cuts. Line 70 represents the opening of the passageway bed cut at the surface of the cornea. The opening 70 has a physical width which is narrower than the diameter of the central bed cut 34, but is large enough to allow the small, unexpanded lens or strengthening material to pass therethrough. The passageway bed cut has its shape defined by a femtosecond laser or some future developed laser which has the same ability and which has had its software modified to cut the shape defined by lines 64, 66, 68 and 70. The shape of the passageway bed cut can also be defined by a mask or coating on the glass of the Intralase patient interface which defines the desired truncated triangle shape. The end cut 70 is cut with an A femtosecond laser or some future developed laser which has the same ability so as to go down from the surface of the cornea to the level of the passageway bed cut which is at the same as the level of the central bed cut. The lens or strengthening material 62 is inserted into the opening 70 and pushed through the passageway bed cut into the central bed cut 34. The lens or strengthening material then expands and is trapped in the central bed cut such that it is restrained from movement by the walls of the central bed cut and the narrower width of the passageway bed cut.

FIG. 13 is another sample using this same idea of a lens or strengthening material which expands when it absorbs water. The central bed cut 34 is formed in the same way as previously described, typically using a femtosecond laser or some future developed laser which has the same ability and operating in spiral cut mode. The laser is used to cut a circular central bed cut having the desired diameter of the implant 62. The passageway bed cut 80 is defined in outline by lines 72, 74, 76 and opening 78. This shape for the passageway bed cut will be referred to as the keyhole cut in the claims. Preferably, the passageway bed cut is at the same level as the central bed cut 34 in the stroma layer, but in other examples, the passageway bed cut can angle down through the stroma layer from the surface of the cornea and terminate somewhere within the perimeter of the central bed cut or on its perimeter (line 74) so as to form a passageway into the central bed cut portion of the pocket. The passageway bed cut is an elongated rectangle having a width along the x axis which is wide enough to accommodate the implant 62 but not wide enough to allow the expandable material to escape back out of the central bed cut after it expands. Lines 72 and 76 are not side cuts and only line 78 represents an opening at the surface of the cornea.

In the example of FIG. 13, a small, rectangular microkeratome can be used to make the passageway bed cut and opening 78. However, a femtosecond laser or some future developed laser which has the same ability and which is suitably programmed to make the shape of the passageway bed cut may also be used to make the passageway bed cut.

FIG. 15 is a diagram of a microkeratome suitable to make the passageway bed cut 80 in FIG. 13. A sharp microkeratome 92 is used having the desired shape and dimensions for the keyhole passageway bed cut 80 is supported in the middle of a frame or web 98 with two sled runners 96 and 94. The shape and dimensions of the blade 92 match with the desired width of the passageway bed cut and the microkeratome has a rounded tip will be referred to in the claims as the “keyhole passageway bed cut blade” shape. The sled runners are conventionally designed sled runners like are used for all microkeratomes. The sled runners slide along a surface of the suction device which attaches to the surface of the cornea to stabilize it. This stabilizes the microkeratome so that it can be slide toward the cornea and slice into the stroma layer to form a passageway bed cut 80 which joins the central bed cut 34.

Likewise, a microkeratome having the width of the passageway bed cut in the example of FIG. 11 can be used to form the passageway bed cut in the examples of FIGS. 11 and 14B instead of a femtosecond laser or some future developed laser which has the same ability. This passageway bed cut will join the central bed cut 34 which is formed by a femtosecond laser or some future developed laser which has the same ability and is operated in spiral cut mode.

A microkeratome suitable to make the passageway bed cut in the examples of FIGS. 11 and 14B is shown in FIG. 16. The blade 100 has a rectangular body having a width W equal to the distance between cut boundaries 54 and 56 in FIG. 11 or 86 and 84 in FIG. 14B and having a rounded tip 102 having the diameter of the desired implant and the central bed cut 34 in FIGS. 11 and 14B.

After the lens or strengthening material 52 is inserted into the central bed cut, the epithelium grows back quickly and seals the opening 60 in FIGS. 11 and 88A and 88B in FIGS. 14A and 14B.

The preferred material at the present time to make the implant 52 in the various non-expandable lens examples is the double network hydrogel developed by Stanford University. This material has a tensile strength of 7.0 MPa, a water content of 80%, a glucose permeability of 1.0 E-6 cm²/s and a mesh size of 25 angstroms. This material has high strength and elasticity for ease of implantation and durability. Its high water content means it is highly permeable. It is important that the material of the implanted lens or strengthening material be permeable to glucose so that nutrients can reach the epithelium from the aqueous humor. It is also important that the material of the lens or strengthening material be permeable to air so that air contacting the surface of the cornea can diffuse down to the lower layers of the cornea which are not fed by blood vessels (so that they can remain transparent).

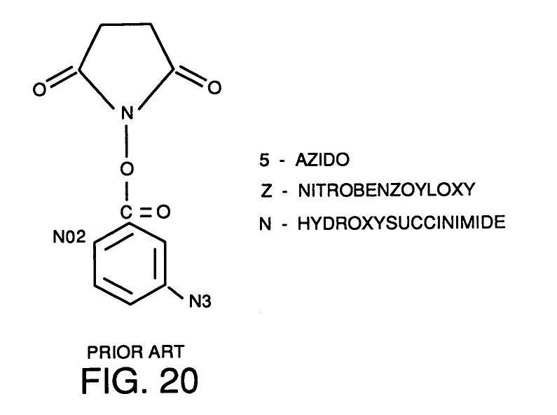

The expandable lens or strengthening material for implant 62 used in the examples of FIGS. 12 and 13 is available from the Stanford University Corneal Product Development team or licensees thereof which have a license to make the swellable Intraocular Lens (IOL) material. This material is a double network hydrogel. It has 80% water content and dramatically enhanced mechanical strength compared to other hydrogels. The material has a high refractive index, is well characterized and already approved by the Food and Drug Administration and exhibits rapid swelling when exposed to the water in the stroma layer surrounding the central bed cut. The first network is poly(ethylene glycol) having the chemical structure of FIG. 18 where n indicates a repeating group. The second network is poly (acrylic acid) having the chemical composition of FIG. 19. The material has a unique surface chemistry for protein and cellular adhesion having the chemical structure shown in FIG. 20.

Stanford envisions use of this material for corneal onlays in humans for reversible, minimally-invasive alternatives to LASIK with no ablation of corneal tissue. Such a use may not require creation of a corneal flap since the onlay is placed on the surface of the cornea and the epithelium grows over it to hold it in place. In this prior art procedure, the epithelium is removed and the polymeric lenticule is placed on the stroma layer. The epithelium is then allowed to regenerate over the lenticule. The procedure has already been done in rabbits, but the applicants believe it has not yet been done in humans.

The primary product application of the double network hydrogel envisioned by Stanford for human use is as a corneal inlay. In this prior art procedure (applicants believe it has only been done in rabbits to date), a corneal flap is created using prior art LASIK procedures which include side cuts and a cut at one end of the flap so that the flap can be lifted up and back. The hydrogel lenticule is then placed on the stroma layer and the flap is laid back down. Surface tension alone apparently holds the lenticule in place where it is initially spotted, so if the eye suffers a blow before the epithelium regenerates over the flap and seals the side cuts and end cuts, the operation may need to be repeated.

Stanford envisions use of this double network material for intrastromal implants, swellable intraocular lens, intralens implants and artificial corneas. Stanford filed two patent applications covering corneal inlays and corneal onlays in 2005 and has one provisional patent application on swellable intraocular lens applications. Stanford has one issued U.S. Pat. No. 6,976,997, covering artificial cornea implants, and methods for making and using such implants, which is hereby incorporated by reference.

Another material that may possibly be used for the implant 52 is acrylic such as is used for present day contact lenses. Acrylic lens are permeable to gas, but may or may not be sufficiently permeable to glucose to be implanted within the cornea as opposed to being placed on top of the cornea as is the case of a contact lens. It is possible that an acrylic lens that is small enough for glucose to go around it may suffice to practice the invention.

Although the invention has been disclosed in terms of the preferred and alternative examples disclosed herein, those skilled in the art will appreciate that modifications and improvements may be made without departing from the scope of the invention. All such modifications are intended to be included within the scope of the claims appended hereto.

What is claimed is:

1. A surgical procedure to implant a lens or strengthening material inside the stroma layer of a cornea, comprising the steps:
forming a pocket in the stroma layer of a cornea; and
slipping a lens or strengthening material into said pocket.

2. The procedure of claim 1 wherein said pocket is formed using a LASIK laser which has been adapted to make a central bed cut having the desired shape and a passageway bed cut having an opening to the surface of said cornea.

3. The procedure of claim 1 wherein said pocket is formed using a microkeratome which has been modified to form the desired shape and size for a central bed cut, a passageway bed cut and an opening which define said pocket.

4. The procedure of claim 1 wherein said pocket is formed by performing the following steps:
1) forming a central bed cut using a femtosecond laser or future developed laser having the same capability of said femtosecond laser, said laser being programmed to cut a circular bed cut having a desired diameter; and
2) forming a passageway bed cut which joins said central bed cut and which has an opening at the surface of said cornea.

5. The procedure of claim 4 wherein said central bed cut is cut by said laser operating in spiral cut mode, and wherein said passageway bed cut is formed using a femtosecond laser or future developed laser having the same capability of said femtosecond laser, said laser having been modified to cut the desired shape and size for the passageway bed cut.

6. The procedure of claim 4 wherein said passageway bed cut is formed using a microkeratome which has been modified to cut the desired shape and size for said passageway bed cut.

7. The procedure of claim 5 wherein said passageway bed cut is cut to be narrower in width than said central bed cut and wherein said lens or strengthening material (hereafter implant) is an expandable hydrogel which is small enough to fit through said opening of said passageway bed cut and be inserted into said central bed cut and wherein said implant is of a material which expands after insertion into said central bed cut to occupy all or most of the area of said central bed cut so as to be retained in place thereby.

8. The procedure of claim 6 wherein said passageway bed cut is cut to be narrower in width than said central bed cut and wherein said lens or strengthening material (hereafter implant) is an expandable hydrogel which is small enough to fit through said passageway bed cut and be inserted into said central bed cut and is of a material which expands after insertion into said central bed cut to occupy all or most of the area of said central bed cut and be retained in place thereby.

9. The procedure of claim 4 wherein said central bed cut is cut using a femtosecond laser or future developed laser having the same capability of said femtosecond laser, said laser being adapted to cut a circular central bed cut in the said stroma layer having a diameter sufficient to accommodate the size of lens or strengthening material to be inserted, said laser using a spiral cut mode, and wherein said passageway bed cut is cut to have a predetermined shape.

10. The procedure of claim 9 wherein said predetermined shape is a passageway having a width equal to the diameter of said central bed cut.

11. The procedure of claim 10 wherein said passageway bed cut is cut in the said stroma layer at the same level as said central bed cut, and wherein most or all of the passageway bed cut is in the same plane as said central bed cut.

12. The procedure of claim 11 wherein said passageway bed cut is cut with a femtosecond laser or future developed laser having the same capability of said femtosecond laser, said laser having been adapted to cut the desired shape of said passageway bed cut.

13. The procedure of claim 11 wherein said passageway bed cut is cut with a microkeratome which has been modified to cut said passageway bed cut with no side cuts only an opening.

14. The procedure of claim 4 wherein said central bed cut is cut using a femtosecond laser or future developed laser having the same capability of said femtosecond laser operating in spiral cut mode, and wherein said passageway bed cut is cut using a femtosecond laser or future developed laser having the same capability of said femtosecond laser adapted to make a truncated triangle shape cut with an opening at the surface of said cornea and no side cuts, and wherein said step of inserting said lens or strengthening material into said pocket comprises inserting an expandable hydrogel lens or strengthening material which expands after contact with water so as to occupy all or most of the space of said central bed cut.

15. The procedure of claim 4 wherein said central bed cut is cut using a femtosecond laser or future developed laser having the same capability of said femtosecond laser, said laser operating in spiral cut mode, and wherein said passageway bed cut is cut using a femtosecond laser or future developed laser having the same capability of said femtosecond laser programmed to make a keyhole shaped cut with an opening at said surface of said cornea or using a microkeratome adapted to make said keyhole shaped cut with an opening at said surface of said cornea but no side cuts, and wherein said step of inserting said lens or strengthening material into said pocket comprises inserting an expandable hydrogel lens or strengthening material which expands after contact with water so as to occupy all or most of the space of said central bed cut.

16. A surgical procedure to implant a lens or strengthening material inside the stroma layer of a cornea, comprising the steps:
forming a pocket in the stroma layer of a cornea by performing the following steps:
making a central bed cut using a femtosecond laser or future developed laser having the same capability of said femtosecond laser, said laser operating in spiral cut mode and adapted to make a circular cut having a diameter sufficient to accommodate a circular lens or strengthening material (hereafter implant);
making a passageway bed cut using a femtosecond laser or future developed laser having the same capability of said femtosecond laser or microkeratome, said laser or microkeratome adapted to make a desired shape for said passageway bed cut with no side cuts and an opening at the surface of said cornea that provides a place into which a lens or strengthening material may be inserted, said passageway bed cut joining said central bed cut such that said lens or strengthening material may be inserted into said central bed cut using said passageway bed cut as a pathway; and
slipping a lens or strengthening material into said pocket.

17. The surgical procedure of claim 16 wherein said lens or strengthening material is a double network hydrogel which is flexible, permeable to oxygen and glucose and has adequate physical strength to survive the procedure and normal wear and tear on the cornea.

18. A surgical procedure to implant a lens or strengthening material inside the stroma layer of a cornea, comprising the steps:
forming a pocket in the stroma layer of a cornea by performing the following steps:
making a central bed cut using a femtosecond laser or future developed laser having the same capability of said femtosecond laser, said laser operating in spiral cut mode and adapted to make a circular cut having a diameter sufficient to accommodate a circular lens or strengthening material;
making a passageway bed cut using a femtosecond laser or future developed laser having the same capability of said femtosecond laser, said laser adapted to make a truncated triangle shaped bed cut with no side cuts and an opening at the surface of the cornea into which a lens or strengthening material may be inserted, said passageway bed cut joining said central bed cut such that said lens or strengthening material may be inserted into said central bed cut using said passageway bed cut as a passageway; and
slipping an expandable lens or strengthening material into said pocket.

19. The process of claim 18 wherein said expandable lens or strengthening material is a double network hydrogel which is flexible, permeable to oxygen and glucose and has adequate physical strength to survive the procedure and normal wear and tear on the cornea.

20. A surgical procedure to implant a lens or strengthening material inside the stroma layer of a cornea, comprising the steps:
forming a pocket in the stroma layer of a cornea by performing the following steps:
making a central bed cut using a femtosecond laser or future developed laser having the same capability of said femtosecond laser, said laser operating in spiral cut mode and adapted to make a circular cut having a diameter sufficient to accommodate a circular lens or strengthening material;
making a passageway bed cut using a femtosecond laser or future developed laser having the same capability of said femtosecond laser or microkeratome blade, said laser or blade adapted to make a keyhole shaped passageway bed cut with no side cuts and an opening at the surface of said cornea into which a lens or strengthening material may be inserted, said passageway bed cut joining said central bed cut such that said lens or strengthening material may be inserted into said central bed cut using said passageway bed cut as a pathway; and
slipping an expandable lens or strengthening material into said central bed cut through said passageway bed cut.

21. The process of claim 20 wherein said expandable lens or strengthening material is a double network hydrogel which is flexible, permeable to oxygen and glucose and has adequate physical strength to survive the procedure and normal wear and tear on the cornea.

22. A surgical procedure to implant a lens or strengthening material inside the stroma layer of a cornea, comprising the steps:
forming a pocket in the stroma layer of a cornea using a microkeratome or a femtosecond laser or future developed laser having the same capability of said femtosecond laser, said laser or microkeratome adapted to form a single bed cut having a rounded shirt pocket shape with no side cuts and a single opening to the surface of said cornea; and
slipping a lens or strengthening material into said pocket through said opening.

23. A modified microkeratome blade comprising:
a web or frame portion;
a blade portion attached to said web or frame portion, said blade having a predetermined shape and dimensions so as to cut a pocket or a passageway bed cut in a cornea with no side cuts and a single opening at the surface of a cornea;
first and second sled runners attached to said web or frame portion and on either side of said blade portion but separated therefrom.

24. The blade of claim 23 wherein said predetermined shape and dimensions of said passageway bed cut are the shape and dimensions of a keyhole cut shaped passageway bed cut.

25. The blade of claim 23 wherein said predetermined shape and dimensions are the shape and dimensions of said pocket are a rounded shirt pocket shape.

26. A modified patient interface, comprising:
a transparent eye contact plate of material which is rigid enough to deform a cornea to relatively flat shape when said plate is brought into contact with the cornea;
a rigid frame attached to the periphery of said transparent eye contact plate;
a set of legs attached to said frame and extending away from said transparent plate;
an upper ring frame attached to said legs; and
a mask on said eye contact plate which is opaque to laser energy output by an a femtosecond laser or some future developed laser which has the same ability, said mask having a transparent area which defines the desired shape of a bed cut to be made in said cornea.

27. The patient interface of claim 26 wherein said desired shape is a passageway bed cut shape.

28. The patient interface of claim 26 wherein said desired shape is a keyhole cut passageway bed cut.

29. The patient interface of claim 26 wherein said desired shape is a truncated triangle passageway bed cut shape.
